(19) United States
(12) Patent Application Publication
(10) Pub. No.: US 2020/0397606 A1
(43) Pub. Date: Dec. 24, 2020

(54) HAND ORTHOSIS AND METHOD FOR ADAPTING A HAND ORTHOSIS

(71) Applicant: Ottobock SE & Co. KGaA, Duderstadt (DE)

(72) Inventors: Guido KAHLMEYER, Siemerode (DE); Julia BIALOWONS, Göttingen (DE); Klaus LIDOLT, Duderstadt (DE); Alexander HARDT, Aßlar (DE); Johan Fredrik FLOOD, Katrineholm (SE)

(73) Assignee: Ottobock SE & Co. KGaA, Duderstadt (DE)

(21) Appl. No.: 15/733,123

(22) PCT Filed: Nov. 16, 2018
(86) PCT No.: PCT/EP2018/081629

§ 371 Date: May 21, 2020

(30) Foreign Application Priority Data
Nov. 24, 2017 (DE) 10 2017 127 890.7

(51) Int. Cl. A61F 5/01

(52) U.S. Cl. CPC A61F 5/0118

(57) ABSTRACT

A hand orthosis having a cuff for fixing the hand orthosis to a wrist joint or a lower arm of a wearer of the hand orthosis, at least one carrier element, and at least one hand support element that can be fixed to the carrier element. The hand support element includes a contour that is designed to be symmetrical to a longitudinal axis of the hand support element and includes a thumb support area on both sides of the longitudinal axis.

The invention relates to a hand orthosis with a cuff for fixing the hand orthosis to a wrist joint or a lower arm of a wearer of the hand orthosis, at least one carrier element and at least one hand support element, which can be fixed to the carrier element. The invention also relates to a hand support element for such a hand orthosis and a method for adapting such a hand orthosis.

Hand orthoses have been known within the scope of the prior art for many years. They feature a cuff with which the hand orthosis is arranged on the wrist or the lower arm of the wearer. The design of this cuff is different depending on the intended purpose of the hand orthosis. There are cuffs which only extend over the wrist and a small part of the lower arm so that the supporting effect remains small, for example if only a thumb is to be supported. If however the wrist itself is to be immobilized, a larger cuff is required which can also comprise rigid or simply less flexible elements, such as splints or rods.

In particular, hand orthoses can be used to support the surface of the hand and, where appropriate, the fingers of a hand by way of a hand support element and, for instance, to prevent spasticities. In this case, such a hand support element is preferably arranged on the palm of the hand, i.e. volarly, and the hand rests on the hand support element. Of course, it is also possible to arrange the hand support element on the opposite side of the hand, i.e. dorsally, such that the hand is fixed to the hand support element in order to support it.

The disadvantage is that a multitude of different hand orthoses must be available. As such, different sized hand support elements must be available for different sized hands. If the hand support element is considerably larger than the hand to be supported, it is possible that the hand support element will bump objects, such as doors, cupboards or tables, if careless hand movements are made. This results in the transfer of impacts and vibrations to the hand which is actually meant to be supported and possibly immobilized, which is unpleasant, potentially painful and, from a therapeutic point of view, undesirable. Conversely, if the hand support element is significantly smaller than the hand to be supported, the necessary supporting effect cannot be achieved to the full extent since, for instance, the last digits can no longer rest on the hand support element, such that these digits are not supported.

Furthermore, different hand support elements must be provided for left and right hands.

In addition, the prior art specifies different hand support elements for the varying degrees of freedom of movement that should still be enabled by the hand support element and the hand orthosis. If the hand should generally remain freely moveable, it is enough to provide a hand support element on which the hand loosely rests, for instance. The hand support element is arranged on the hand via the cuff such that is cannot become detached, wherein said cuff serves the arrangement of the hand orthosis on the wrist joint or the lower arm of the wearer, and wherein the hand itself need not be fixed to the hand support element.

Conversely, if the freedom of movement of the hand is to be considerably more restricted, for example to prevent spasticities, it may be beneficial to fix the entire hand, each individual finger or just certain fingers to the hand rest element. Given that various fixing elements, especially belts, have to be provided for this purpose, it is also necessary in these cases to provide different hand orthoses with different hand support elements.

This is disadvantageous because a large quantity of different hand orthoses must be produced and provided, which increases the price of warehousing.

The invention thus aims to remedy these disadvantages or at least to reduce them.

The invention solves the problem by way of a hand orthosis with the properties described in the generic term in claim 1, characterized in that the hand support element has a contour that is designed to be symmetrical to a longitudinal axis and features a thumb support area on both sides of the longitudinal axis. The hand orthosis according to the invention consequently features a hand support element with two thumb support areas. As a result, the hand support element can be used as a support element for both a right hand and a left hand. Therefore, it is no longer necessary to provide different hand support elements or even different hand orthoses for left and right hands. If the hand orthosis is to be adapted to a right or left hand, the thumb element that is not required is removed, for example cut off with a pair of scissors. Of course, other cutting tools may be used. Where appropriate, the cutting edges are polished and deburred in order to reduce the risk of injury and to make it more comfortable to wear.

Preferably, the hand support element features markings which represent the stylized contours of right and/or left hands of different sizes. In this regard, in order to adapt the hand orthosis to the hand of the wearer of the hand orthosis, the hand support element can also be trimmed, shortened or adjusted to fit. To this end, the orthopedic technician or another person who adapts the hand orthosis, for example, selects a suitable contour of a right or left hand and trims the hand support along this contour. In this way, the hand support element is both adapted to the side of the hand that is to be treated, i.e. a right hand or a left hand, and cut to the size of the respective hand. In this case, it may also be practical to deburr or smooth any cutting edges which may occur.

The markings preferably feature indentations and/or openings. This renders the cutting along the contours and markings easier. If they are indentations, the thickness of the respective hand element at the points of these markings is low, so that it is considerably easier to cut, for example with a pair of scissors. Conversely, if the markings already feature openings, it is no longer necessary to make a cut at these points; rather, it is only necessary to cut through webs which extend along the marking between the individual openings. This also renders cutting much easier.

Preferably, the hand support element comprises several, preferably three, four or five openings, which extend parallel or almost parallel to the longitudinal axis of the hand support element. These openings are preferably arranged in such a way that they are arranged between the contact surfaces for the individual fingers of a hand that is to be supported by the hand support element. Belts, straps or other fixing elements can be passed through them to fix individual fingers, groups of fingers or the entire hand to the hand rest element. The belts can be made from an elastic or a non-elastic material and are preferably equipped with fastening elements, such as clasps, velcro elements or press studs, in order to ensure that the respective body part can be fastened and fixed on the hand support element. Preferably, the fixing elements can be removed from the hand support element, for example in order to replace them with other elements if, for instance, the length of the fixing elements is to be adapted or the fixing elements or hand support element cleaned or repaired.

The hand support element is preferably made from a plastic. This results in a pleasant feel and a low weight as well as rendering the hand support element easier to cut through, especially along the markings. The hand support element is also preferably water-resistant.

In a preferred configuration, the hand support element features at least one fixing element, preferably a positive-locking element, on the side that faces towards the carrier element, wherein said fixing element is configured to interact with a corresponding fixing element or positive-locking element arranged on the carrier element or with the carrier element itself. It is therefore possible to fix the hand support element to the carrier element, in particular in a positive-locking manner. For example, a clip connection means that the fixing can be easily released. The hand support element can preferably be fixed to the carrier element such that it can be detached. As a result, it is no longer necessary to connect or fix the hand support element to the carrier element as early as the production phase. Rather, the hand support element can be arranged by the orthopedic technician, for example, or possibly even by the patient himself at the desired point and in the desired orientation and position on the carrier element. This also reduces warehousing requirements and increases flexibility and applicability.

The invention also solves the problem by way of a hand support element for a hand orthosis as described here. The invention also solves the problem by way of a method for adapting a hand orthosis according to an example of an embodiment of the present invention, wherein the method includes the detachment, preferably the cutting off, of a thumb support area of the hand support element. The cutting edges can then preferably be smoothed, deburred or subjected to another type of post-treatment so as to reduce the risk of injury and render it more comfortable to wear. Preferably, the hand support element is cut to size along at least one of the markings.

In the following, an example of an embodiment of the present invention will be explained in more detail by way of the attached figures: They show

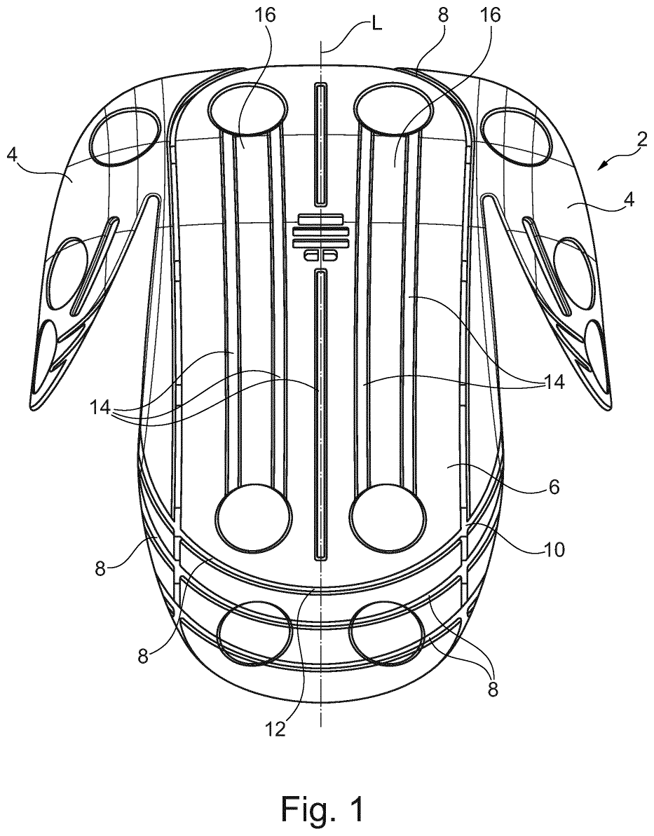

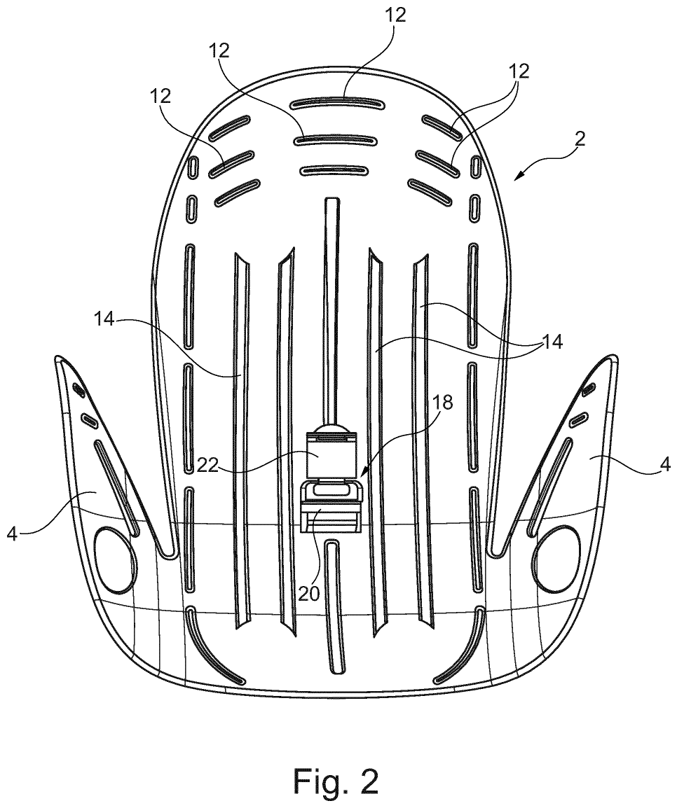

FIGS. 1 and 2—schematic depictions of a hand support element from different perspectives and

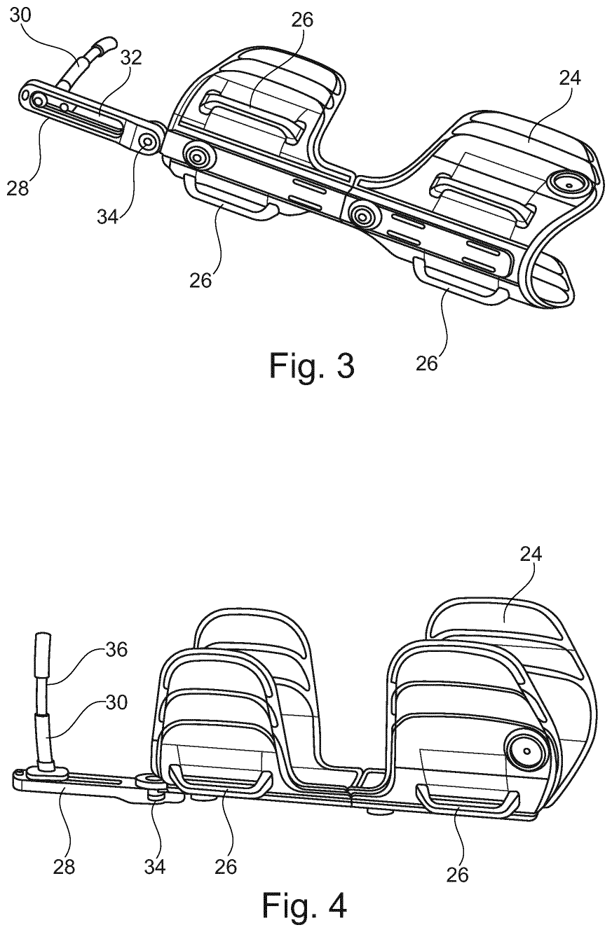

FIGS. 3 and 4—schematic representations of a part of a hand orthosis.

FIG. 1 depicts a hand support element 2 for a hand orthosis according to a first example of an embodiment of the invention. The hand support element features two thumb support areas 4 and is designed to be symmetrical relative to its longitudinal axis L. A hand contact surface 6 features several markings 8, which are designed in the form of indentations 10 and openings 12. To cut the hand support element 2 shown to size, one of the two thumb support elements 4 is removed. If the hand support element 2 is to be used for a right hand, the thumb support area 4 shown on the left in FIG. 1 is removed. If the hand support element 2 is to be used for a left hand, the thumb support area 4 shown on the right in FIG. 1 is removed. Depending on the size of the hand, the hand support element 2 is cut to size along one of the markings 8 shown.

The hand support element 2 shown also features slits 14, through which, for example, belts or other fixing elements can be guided and fixed to the hand support element. In this way, it is possible, for instance, to fix individual fingers of a hand to a web 16, which is bordered on both sides by a slit. Of course, it is also possible to guide a belt or strap through two non-adjacent slits 14 and thereby fix several fingers, all fingers and/or the palm of the hand to the hand support element 2.

FIG. 2 shows the hand support element 2 from FIG. 1 from a different perspective. It is now shown from below, whereas the representation in FIG. 1 is from above. The two thumb support areas 4, the openings 12 and the slits 14 can also be seen from this underside. In the center is a fixing element 18, which in this case comprises a first positive-locking element 20. This is designed in the form of a groove and can be locked by way of a lock 22, which is arranged such that it can be swivelled. With this fixing element 18, the hand support element 2 can be fixed to a carrier element, not depicted in FIGS. 1 and 2.

FIGS. 3 and 4 show further components of a hand orthosis according to an example of an embodiment of the present invention. Loops 26 are arranged on a lower arm shell 24, which is made of plastic for example, through which belts or straps can be guided; the lower arm shell 24 is arranged on a lower arm of the wearer using said belts or straps. On the left-hand side of FIGS. 3 and 4, which is assigned to the hand when a hand orthosis is applied, there is a support brace 28 on which a carrier element 30 is arranged. The carrier element 30 is arranged on an elongated hole 32 in the support brace 28 such that it can be displaced. It can also be arranged in two different orientations. Specifically, in FIG. 3 it is clear that the carrier element 30 is designed to be slightly curved. While in FIGS. 3 and 4 a part of a hand orthosis is designed for a left hand, a simple turning or reconnection of the carrier element 30 can adapt the component to fit a hand orthosis for a right hand.

The support brace 28 is arranged on the lower arm shell 24 via an angle adjustment unit 34, such that an angle at this point can be adjusted.

Specifically, in FIG. 4 it is clear that the carrier element 30 features a taper 36, in which it has a reduced diameter. This is advantageous but not absolutely necessary. The diameter and cross-section of this taper 36 of the carrier element 30 is adapted to the first positive-locking element 20 on the hand support element 2, as shown in FIG. 2, for example. As a result, the hand support element 2 can be arranged on the carrier element 30 in a simple and detachable manner.

### REFERENCE LIST

L longitudinal axis
2 hand support element
4 thumb support area
6 hand contact surface
8 marking
10 indentation
12 opening
14 slit
16 web
18 fixing element
20 first positive-locking element
22 lock
24 lower arm shell
26 loop
28 support brace
30 carrier element
32 elongated hole
34 angle adjustment unit
36 taper

1. A hand orthosis comprising:
a cuff for fixing the hand orthosis to a wrist joint or a lower arm of a wearer of the hand orthosis;
at least one carrier element;
at least one hand support element, which can be fixed to the at least one carrier element;
wherein the at least one hand support element includes a contour that is designed to be symmetrical to a longitudinal axis of the at least one hand support element and includes a thumb support area on both sides of the longitudinal axis.

2. The hand orthosis according to claim 1, wherein the at least one hand support element features includes markings which represent stylized contours of right and left hands of different sizes.

3. The hand orthosis according to claim 2, wherein the markings include at least one of indentations and openings.

4. The hand orthosis according to claim 1, wherein the hand support element comprises plastic.

5. The hand orthosis according to claim 1, wherein the at least one hand support element comprises at least one fixing element on a side that faces the at least one carrier element, wherein the fixing element is configured to interact with a corresponding fixing element or positive-locking element arranged on the at least one carrier element or with the at least one carrier element itself.

6. A hand support element for a hand orthosis according to claim 1.

7. A method for adapting a hand orthosis according to claim 1, wherein the method comprises removing a thumb support area of the at least one hand support element.

8. The method according to claim 7, wherein the at least one hand support element includes markings which represent stylized contours of right and left hands of different sizes, and the at least one hand support element is cut to size along at least one of the markings.

9. The method according to claim 7, wherein removing the thumb support area includes cutting off the thumb support area.

10. The hand orthosis according to claim 5, wherein the at least one fixing element includes a positive-locking element.

11. A hand orthosis comprising:
a cuff configured to fix the hand orthosis to a wrist joint or a lower arm of a wearer of the hand orthosis;
at least one carrier element;
at least one hand support element fixed to the at least one carrier element and comprising:
a contour designed to be symmetrical with a longitudinal axis of the at least one hand support member;
a thumb support area arranged on both sides of the longitudinal axis.

12. The hand orthosis according to claim 11, wherein the at least one hand support element includes markings that represent stylized contours of right hands and left hands of different sizes.

13. The hand orthosis according to claim 12, wherein the markings include at least one of indentations and openings.

14. The hand orthosis according to claim 11, wherein the at least one hand support element comprises plastic.

15. The hand orthosis according to claim 11, wherein the at least one hand support element comprises:
at least one fixing element on a side that faces the at least one carrier element;
a fixing element configured to interact with a corresponding fixing element or positive-locking element arranged on the at least one carrier element or with the at least one carrier element itself.

16. The hand orthosis according to claim 15, wherein the at least one fixing element includes a positive-locking element.
